(19) United States
(12) Patent Application Publication
(10) Pub. No.: US 2003/0219461 A1
(43) Pub. Date: Nov. 27, 2003

(54) Parenteral combination therapy for infective conditions

(76) Inventors: Nancy J. Britten, Portage, MI (US); Niki A. Waldron, Kalamazoo, MI (US); Thomas J. Yellig, Gobles, MI (US); Ching-Chiang Su, Kalamazoo, MI (US)

Correspondence Address: PHARMACIA CORPORATION, GLOBAL PATENT DEPARTMENT, POST OFFICE BOX 1027, ST. LOUIS, MO, 63006, US

(21) Appl. No.: 10/393,267

(22) Filed: Mar. 20, 2003

Related U.S. Application Data
(63) Continuation-in-part of application No. 09/948,827, filed on Sep. 7, 2001.
(60) Provisional application No. 60/231,767, filed on Sep. 12, 2000.

(51) Int. Cl. A61K 39/12; A61K 38/00

(52) U.S. Cl. 424/204.1; 514/2

(57) ABSTRACT

A method is provided for treatment or prevention of an infective condition having an inflammatory component. The method comprises parenteral administration of an antibacterial agent in an antibacterially effective amount, in combination therapy with a selective cyclooxygenase-2 inhibitor in an amount sufficient to provide systemic anti-inflammatory activity. Also provided is a parenterally deliverable pharmaceutical composition comprising an antibacterial agent and a selective COX-2 inhibitor together with one or more excipients.

This application is a continuation in part of U.S. application Ser. No. 09/948,827, filed on Sep. 7, 2001, which claims priority of U.S. provisional application Serial No. 60/231,767, filed on Sep. 12, 2000.

### FIELD OF THE INVENTION

The present invention relates to a method of treatment of a non-ocular infective condition having an inflammatory component. The invention also relates to a pharmaceutical composition suitable for parenteral administration for treatment of such a condition, and to a process for preparing such a composition.

### BACKGROUND OF THE INVENTION

Many infective conditions have an associated inflammatory component. Inflammation is generally a localized protective response to an injurious agent that serves to destroy, dilute or wall off the agent and the injured tissues. Frequently, however, such inflammation becomes acute, and various organs and systems of a subject having an infective condition suffer from effects of the inflammation itself, for example swelling, pain, heat, redness, increased mucous or mucous/catarrhal secretions, loss of function and the like, as well as a general symptomology characterized by fever, anorexia and sensory dulling. Damage caused by associated inflammation is sometimes more serious than that due to the infection itself. In addition to its detrimental effect on tissues, inflammation associated with an infection can also inhibit an antibacterial agent from effectively reaching the site of infection, thereby diminishing the ability of that antibacterial agent to treat the infection.

Very few antibacterial agents possess anti-inflammatory, antipyretic or analgesic properties in addition to their antibacterial activity. Therefore, treating an infective condition with an antibacterial agent alone typically does not alleviate the inflammation, pain, swelling, fever and other complications that often accompany such an infective condition. These problems are usually not totally resolved until the causal organism of the infective condition has been eliminated or reduced to a subpathogenic population by the antibacterial agent.

Treatment of an infective condition having an inflammatory component with an anti-inflammatory agent alone can reduce inflammation, swelling, pain, fever and other complications, but does not treat the underlying infective condition.

Parenteral methods of administration are an important option for delivery of therapeutic agents. Many antibacterial agents but relatively few nonsteroidal anti-inflammatory drugs (NSAIDs) are commercially available in injectable form. Non-selective NSAIDs such as ketorolac tromethamine salt that are available for parenteral use are effective analgesics but have been associated with side effects typical of such non-selective NSAIDs, for example gastrointestinal irritation and toxicity, upper gastrointestinal ulceration and bleeding, renal toxicity, blockage of platelet aggregation and hepatic damage. Serious side effects are also associated with other commonly used anti-inflammatory agents such as corticosteroids. Corticosteroid use can produce hypertension, arteriosclerosis, diabetes, hyperglycemia, osteoporosis, electrolyte imbalance, slow healing of infections, elevated blood cholesterol, detrimental effects on the functioning of both cellular and humoral defense mechanisms, pituitary-adrenal suppression, fluid and salt retention that can aggravate heart or kidney disease, and increased incidences of cataracts and glaucoma. Such side effects have seriously limited the use of parenteral formulations of non-selective NSAIDs and of corticosteroids.

The use of antibacterial agents and the use of anti-inflammatory agents for prevention and treatment of a variety of disease states are well known.

U.S. Pat. No. 5,756,529 to Isakson & Talley discloses a method of using pyrazolyl benzenesulfonamide compounds to treat inflammation in a companion animal. Such compounds are said to be useful for treatment of pain, fever, joint disease, traumatic injury, arthritis, myositis, tendinitis, equine colic, mastitis, peritonitis, skin conditions, burns, gingivitis, hypersensitivity, conjunctivitis, eye inflammation, swelling and myocardial ischemia.

International Patent Publication No. WO 99/20259 discloses a combination of thiamphenicol and diclofenac for use in veterinary medicine to treat infections with associated inflammatory conditions.

International Patent Publication No. WO 02/06865 discloses a composition comprising one or more bioactive agents in a non-aqueous carrier, wherein the composition has been adjusted to have a water activity of about 0.2 to about 0.5. Parenteral, topical, oral, intravaginal, rectal and intramammary routes of administration are proposed. Among the bioactive agents listed are anti-infectives, antineoplastics, immunomodulators, antipyretics, analgesics and anti-inflammatory agents (e.g., cyclooxygenase-2 (COX-2) inhibitors).

International Patent Publication No. WO 02/05848 discloses use of COX-2 inhibitors to treat and prevent ocular COX-2 mediated disorders, and indicates that antibiotics, antivirals, anti-infectives and the like can be combined with COX-2 inhibitors to treat ocular disorders. Compositions can be administered orally, parenterally, rectally, topically or by inhalation.

U.S. Patent Application Publication No. 2002/0064547 discloses a liquid polymeric composition for controlled release of a hydrophobic bioactive substance. Such a composition comprises 1-30% of a hydrophobic bioactive substance, 1-20% of a poly(lactide-co-glycolide) with specific ratios of polymers, and a mixture of hydrophilic and lipophilic solvents. The composition can be implanted by intramuscular or subcutaneous injection and is said to form a film encapsulated liquid in situ. Bioactive substances said to be useful therein include fipronil, avermectin, ivermectin, eprinomectin, milbemycin, phenylpyrazole, nodulisporic acid, estradiol benzoate, tremblone acetate, noresthistrone, progesterone, an antibiotic, an oil soluble NSAID (e.g., a COX-2 inhibitor), or combinations thereof.

International Patent Publication No. WO 98/50045 discloses a method for producing a veterinary product comprising bringing a selected long acting antimicrobial formulation into intimate admixture with an anti-inflammatory agent and preparing the admixture for parenteral administration.

International Patent Publication No. WO 2002/0080912 discloses a reconstitutable parenteral composition comprising the water soluble selective COX-2 inhibitor parecoxib, that can be given in combination with opioids and other analgesics.

International Patent Application No. WO 00/38730 discloses a method of treating or preventing neoplasia disorders using a combination of a COX-2 inhibitor and an antineoplastic agent.

U.S. Patent Application Publication No. 2002/0032228 discloses use of a heterocycle containing compound, for example a diphenyl heterocycle derivative, to treat diarrheal diseases, whooping cough, anthrax, smooth muscle contraction conditions and mastitis. Celecoxib and rofecoxib are listed as preferred diphenyl heterocycle derivatives.

All of the above patents and articles are incorporated herein by reference but are not necessarily prior art under patent statutes.

Although the references cited above disclose a number of useful compositions, there still exists a need in the medical field for methods of treatment and prevention for infective conditions having an inflammatory component and for pharmaceutical compositions having one or more of the following advantages over prior art methods and/or compositions: (a) safe, effective treatment and/or prevention for both the infectious and the inflammatory components of a non-ocular infective condition, (b) rapid delivery of therapeutic agent(s) to the site of an infective condition, (c) improvement of the therapeutic index of an active agent while decreasing its general toxicity and minimizing the risk of systemic effects, (d) safe, effective treatment of the pain, inflammation, fever, swelling, heat, redness, increased mucous or mucous/catarrhal secretions, anorexia, sensory dulling, loss of organ or system function, and other complications associated with a non-ocular infective condition, (e) reduction of side effects associated with administration of an antibacterial agent, (i) reduction of side effects associated with administration of an anti-inflammatory agent, (g) efficacy against a wide variety of infectious organisms, (h) sustained release for more convenient and effective prolonged treatment, (i) potential to administer a lower dose of a therapeutic agent while still providing efficacy, and (j) potential to administer a higher dose of an antibacterial agent without increased side effects.

### SUMMARY OF THE INVENTION

Novel methods of treatment and/or prevention and pharmaceutical compositions having some or all of the advantageous attributes described above have now been developed. In particular, there is provided a novel method of treatment and/or prevention of a non-ocular infective condition having an inflammatory component, or an associated complication. The method comprises parenterally administering to a subject an antibacterial agent in an antibacterially effective amount, in combination therapy with a selective COX-2 inhibitor in an amount sufficient to provide systemic anti-inflammatory activity.

The antibacterial agent and the selective COX-2 inhibitor can be administered sequentially in either order or simultaneously. In one embodiment the antibacterial agent and the selective COX-2 inhibitor are administered as a single pharmaceutical composition comprising, in addition to the antibacterial agent and the selective COX-2 inhibitor, a vehicle that comprises at least one pharmaceutically acceptable excipient.

The method is useful for treatment and/or prevention of a non-ocular infective condition having an inflammatory component, and is efficacious in a wide variety of infective conditions involving a wide variety of infectious organisms.

When administered parenterally, the combination therapy of the invention provides enhanced treatment options as compared to administration of either the selective COX-2 inhibitor or the antibacterial agent alone, or compared to administration of the selective COX-2 inhibitor and/or the antibacterial agent by routes other than parenteral.

Combination therapy according to the invention provides effective treatment for both the infectious and the inflammatory components of an infective condition, and can reduce the time required to resolve the infective condition and the associated inflammation.

The term “treatment” herein includes administration parenterally to a subject that does not yet show clinical signs of a non-ocular infective condition having an inflammatory component, but at risk of developing such an infective condition. The invention therefore provides a method for reducing risk of developing an infective condition by a subject at such risk, the method comprising administering to the subject, parenterally, an antibacterial agent in combination therapy with a selective COX-2 inhibitor. For example, combination therapy as described herein can be administered prior to or following surgery to prevent or reduce the risk of a subject developing an infection having an inflammatory component.

In a preferred embodiment, however, combination therapy according to the invention is administered to a subject who has clinical signs of an infective condition having an inflammatory component. The invention therefore provides a method for treating a non-ocular clinical infective condition having an inflammatory component, the method comprising administering to a subject, by parenteral administration, an antibacterial agent in combination therapy with a selective COX-2 inhibitor.

Combination therapy as described herein therefore provides safe, effective treatment for both the inflammatory component as well as the infectious component of a non-ocular infective condition. Such combination therapy can also reduce or alleviate pain, swelling, heat, redness, increased mucous or mucous/catarrhal secretions, anorexia, sensory dulling, loss of function of an organ or system, fever, and/or other complications associated with above described conditions.

While conventional NSAIDs inhibit both isoforms of the cyclooxygenase (COX) enzyme, selective COX-2 inhibitors target COX-2 with minimal to no effect on COX-1, thus effectively reducing inflammation while producing fewer and less severe side effects than those that can occur as a result of treatment with non-selective NSAIDs or corticosteroids. The use of selective COX-2 inhibitors as described herein, rather than non-selective NSAIDs, corticosteroids or other anti-inflammatory agents, provides an effective treatment for inflammation, pain, swelling and fever associated with an infective condition by targeting inflammatory prostaglandins while not affecting constitutive COX-1 derived prostaglandin formation that is required for normal physiological cell functions.

Inflammation associated with an infective condition can inhibit an antibacterial agent from effectively reaching the site of infection. Use of a selective COX-2 inhibitor in combination therapy with an antibacterial agent reduces the inflammation associated with an infective condition and can result in improvement in the ability of the antibacterial agent to effectively reach the site of infection.

Certain antibacterial agents, while being very effective against infective bacteria, are associated with a risk of undesirable side effects, such as transient redness, swelling and inflammation. Acceptable dosages of some antibacterial agents can be practically limited by the need to minimize risk of such side effects. The combination therapy method of the present invention minimizes these risks.

It is believed, without being bound by theory, that certain antibacterial agents, when administered to certain subjects, can promote release of endotoxins that in turn sets off a TNF_{α} (tumor necrosis factor alpha) mediated response, and it is further believed that such response can be blocked or mitigated by the selective COX-2 inhibitor.

Combination therapy according to the invention can enable administration of a lower dose of a therapeutic agent while still providing efficacy.

The parenteral route of administration according to a method of the invention provides, in one embodiment, rapid delivery of the antibacterial agent and the selective COX-2 inhibitor to the site of a non-ocular infective condition. Such rapid delivery provides a faster onset of action of therapeutic agents and is particularly desired for severe infective conditions and/or for conditions having an acute pain component.

In another embodiment parenteral administration can provide sustained release of a selective COX-2 inhibitor and/or an antibacterial agent, resulting in convenient, effective, prolonged treatment of a non-ocular infective condition. For example, sustained release can be provided through injection of a depot formulation.

The invention also provides a parenterally deliverable pharmaceutical composition comprising a pharmaceutically acceptable carrier, an antibacterial agent in an antibacterially effective amount, and a selective COX-2 inhibitor in an amount sufficient to provide systemic anti-inflammatory activity. Such a composition, when administered parenterally, can provide effective treatment for the inflammatory component as well as the infectious component of a non-ocular infective condition having an inflammatory component, and for complications associated with such a condition.

In accordance with this embodiment, a method is provided for effecting rapid systemic delivery of an antibacterial agent and a selective COX-2 inhibitor to treat and/or prevent a non-ocular infective condition having an inflammatory component, the method comprising parenteral administration of such a composition to a subject.

The present invention provides solutions to several long standing problems in the art and possesses one or more advantages over methods and compositions of prior art. Other features, advantages and benefits of the invention will be apparent from the description that follows.

### DETAILED DESCRIPTION OF THE INVENTION

The invention provides a method of treatment and/or prevention of a non-ocular infective condition having an inflammatory component, or a complication associated therewith. The method comprises parenteral administration of an antibacterial agent in an antibacterially effective amount, in combination therapy with a selective COX-2 inhibitor in an amount sufficient to provide systemic anti-inflammatory activity.

A “non-ocular infective condition” herein is a non-neoplastic disease, disorder or condition of a bodily tissue, organ or system other than an eye or part thereof, that is mediated by a pathogenic bacterium or that is otherwise responsive to treatment with an antibacterial agent such as an antibiotic drug, whether or not accompanied by pain, fever, swelling or inflammation. The invention is, however, especially drawn to such conditions having a component of pain, fever, swelling or inflammation.

The term “parenteral administration” herein embraces any means of injection, infusion or implantation of a composition into or through the skin of a subject, regardless of the timescale involved. For example, administration can be by bolus injection that is completed within a very short space of time or by longer-term infusion. Parenteral administration includes, but is not limited to, intravenous, intramuscular, subcutaneous, intradermal, intramedullary, intra-articular, intrasynovial, intraspinal, intrathecal, intracardiac, intraventricular, intracapsular, intracranial and intrasternal administration, as well as conventional and in situ forming implantation techniques and the like. Any known device useful for parenteral injection or infusion of a drug can be used to effect such administration. Parenteral administration herein does not include administration solely to the skin surface, such as topical or transdermal administration. Both the antibacterial agent and the selective COX-2 inhibitor are administered parenterally in the method of the invention. Preferred parenteral administration routes are intravenous, intramuscular and subcutaneous routes.

It will be understood that reference herein to methods involving and compositions comprising “an antibacterial agent” embraces such methods and compositions wherein more than one antibacterial agent is used. Further, more than one selective COX-2 inhibitor can optionally be used.

The term “antibacterially effective amount” as used herein refers to an amount of an antibacterial agent that is sufficient, when administered by the method of the invention, to reduce, relieve, prevent or delay onset of one or more symptoms of an infective condition being treated, or to reduce numbers and/or activity of a causal organism.

The term “amount sufficient to provide systemic anti-inflammatory activity” as used herein refers to an amount of an anti-inflammatory agent, in this case a selective COX-2 inhibitor, that is sufficient, when administered by the method of the invention, to reduce, relieve, prevent or delay onset of one or more symptoms of an inflammatory condition remote from the site of administration. According to the method of the invention, the anti-inflammatory agent enters into the circulatory system and is distributed throughout the body, providing a sufficient concentration of the agent in the bloodstream to provide anti-inflammatory effect at the site or sites of inflammation.

A selective COX-2 inhibitor is a compound that selectively inhibits COX-2 activity. The term “selective COX-2 inhibitor” and “selective cyclooxygenase-2 inhibitor” interchangeably refer to a therapeutic compound that selectively inhibits the COX-2 isoform of the enzyme cyclooxygenase, with less significant inhibition of cyclooxygenase-1 (COX-1). As used herein the term “selective COX-2 inhibitor” also refers to a prodrug or salt that is converted in vivo to a compound that exhibits selective inhibition of COX-2 relative to COX-1. Preferred selective COX-2 inhibitors exhibit a selectivity factor of at least about 10, more preferably at least about 50 and still more preferably at least about 100, wherein “selectivity factor” is defined as IC₅₀(COX-1)/IC₅₀(COX-2), IC₅₀ being the concentration of a compound producing 50% inhibition of enzyme activity in an in vitro or in vivo test.

The term “combination therapy” herein means a treatment regimen wherein the antibacterial agent and the selective COX-2 inhibitor are administered individually or together in such a way as to provide a beneficial effect from co-action of these therapeutic agents. Such beneficial effect can include, but is not limited to, pharmacokinetic or pharmacodynamic co-action of the therapeutic agents. Combination therapy can, for example, enable administration of a lower dose of one or both agents than would normally be administered during monotherapy, thus decreasing risk or incidence of adverse effects associated with higher doses. Alternatively, combination therapy can result in increased therapeutic effect at the normal dose of each agent in monotherapy. Alternatively, combination therapy can maximize the therapeutic effect at higher doses. “Combination therapy” herein is not intended to encompass administration of two or more therapeutic agents as part of separate monotherapy regimens that incidentally and arbitrarily result in sequential or simultaneous treatment.

Administration of the antibacterial agent and the selective COX-2 inhibitor typically is carried out over a defined time period (usually minutes, hours, days or weeks depending upon the combination selected). These therapeutic agents can be administered in a sequential manner, that is, at different times, typically separated by no more than about 24 hours, or in a substantially simultaneous manner.

When administered simultaneously, the antibacterial agent and the selective COX-2 inhibitor can be administered in separate dosage forms or in coformulation, i.e., in a single dosage form. When administered in separate dosage forms, the antibacterial agent is administered parenterally as a pharmaceutical composition comprising said antibacterial agent and a first pharmaceutically acceptable vehicle, and the selective COX-2 inhibitor is administered parenterally as a separate pharmaceutical composition comprising said selective COX-2 inhibitor and a second pharmaceutically acceptable vehicle that can be similar to or different from the first vehicle. The antibacterial agent and the selective COX-2 inhibitor can be administered by the same or different parenteral routes and/or at the same or different sites on the body. In a preferred embodiment, both agents are co-dispersed in the same vehicle and administered in a single operation.

A pharmaceutically acceptable carrier or vehicle is one that has no unacceptably injurious or toxic effect on the subject when administered as a component of a composition by parenteral administration in an amount required herein. No excipient ingredient of such a carrier or vehicle reacts in a deleterious manner with another excipient or with the therapeutic agent(s) in a composition.

A pharmaceutical composition comprising the antibacterial agent and/or the selective COX-2 inhibitor can be a liquid injectable or infusible composition having the agent(s) dispersed in a vehicle as described herein. The term “dispersed” herein means dissolved (i.e., molecularly dispersed) or colloidally dispersed, for example as an emulsion or suspension.

Combination therapy according to the invention can improve the therapeutic index of an active agent by decreasing its general toxicity and minimizing the risk of side effects. Therapeutic index is a measure of the margin between a therapeutically effective dose and a toxic dose of a drug and is typically expressed as the ratio of LD₅₀ (a dose lethal to 50% of a population) to ED₅₀ (a dose therapeutically effective in 50% of the population).

The method according to the invention provides a safe, effective treatment and/or preventive method for non-ocular infective conditions having an inflammatory component, or for an associated complication. Additionally, the method provides an effective treatment option for a wide variety and a large number of infective conditions, as more fully detailed below.

Conditions that can be treated and/or prevented by the method of the invention include, but are not limited to, disorders caused by gram positive organisms such as Staphylococcus, Micrococcus, Streptococcus, Enterococcus, Leuconostoc, Pediococcus, Stomatococcus, coryneform bacteria, Listeria, Erysipelothrix, Kurthia, Bacillus, Nocardia, Rhodococcus, Gordona, Actinomadura, Streptomyces, Mycobacterium, Colostridium, Peptostreptococcus, Propionibacterium, Lactobacillus, Actinomyces and the like; gram negative organisms such as Enterobacteriaceae, Escherichia, Shigella, Salmonella, Klebsiella, Enterobacter, Citrobacter, Serratia, Yersinia, Vibrio, Aeromonas, Plesiomonas, Pseudomonas, Burkholderia, Stenotrophomonas, Ralstonia, Brevundimonas, Comamonas, Acidovorax, Acinetobacter, Achromobacter, Alcaligenes, Moraxella, Methylobacterium, Actinobacillus, Capnocytophaga, Eiknella, Kingella, Legionella, Neisseria, Branhamella, Haemophilus, Bordetella, Brucella, Pasteurella, Bartonella, Afipia, Francisella, Bacteroides, Porphyromonas, Prevotella, Fusobacterium, Campylobacter, Arcobacter, Helicobacter, Leptospira, Leptonema, Chlamydia, Rickettsia, Coxiella, Ehrlichia and the like; and other infective organisms including Treponema, spirochetes, Borrelia, Mycoplasma, Ureaplasma, obligate intercellular bacteria and the like. Infective conditions for which the method of the invention is useful include, without limitation, bacterial soft tissue infections, infections of the respiratory system including upper respiratory tract infections (such as bacterial pneumonia, bronchitis, febrile tracheobronchitis, bacterial laryngotracheitis, pharyngitis, pleuropulmonary infections, tuberculosis, pertussis, purulent nasopharyngitis, lung abscesses, empyenia and the like), sinusitis, ear infections (such as otitis media, otitis externa, acute mastoiditis, otosclerosis, otalgia, Lermoyez's syndrome, Meniere's disease, vestibular neuronitis, benign paroxysmal positional vertigo, herpes zoster oticus, Ramsay Hunt's syndrome, ganglionitis, geniculate herpes, labyrinthitis, purulent labyrinthitis, perilymph fistulas, presbycusis, drug-induced ototoxicity, acoustic neuromas, aerotitis media, infectious myringitis, bullous myringitis, nonchromaffin paragangliomas, chemodectomas, globus jugulare tumors, globus tympanicum tumors, perichondritis, aural eczematoid dermatitis, subperichondrial hematoma, ceruninomas, sebaceous cysts, osteomas, keloids, tympanic membrane infection, tympanitis, otic furuncles, petrositis, conductive and sensorineural hearing loss, epidural abscess, lateral sinus thrombosis, subdural empyema, otitic hydrocephalus, Dandy's syndrome, bullous myringitis, diffuse external otitis, keratosis obturans, otomycosis, acute barotitis media, acute eustachian tube obstruction, postsurgical otalgia, cholesteatoma and the like), otolaryngological infections, infections of the gastrointestinal tract (such as gastroenteritis, helicobacter pylori, bacterial diarrhea, bacillary dysentery, extraintestinal infections, intestinal yersiniosis, enteritis, terminal ileitis, peptic ulcer disease, gastric ulcer disease, atrophic gastritis, mesenteric lymphadenitis, pseudoappendicitis and the like); bacterial meningitis; infection related to abdominal trauma; pyelonephritis; nocardial pulmonary infections (such as pleural effusion, pericarditis, mediastinitis, superior vena cava obstruction and the like); infections of the cardiovascular system including endocarditis, myocarditis and intravascular catheter related infections; synovitis; infections arising from wounds including external wounds, injuries, scalds, bites, sternal wounds, mammaplasty wounds, surgical procedures, etc.; bacteremia, septicemia, acute glomerulonephritis, neonatal infections, diphtheria, intracellular and extracellular bacterial infections; urinary tract infections (such as nongonococcal urethritis, cystitis, urosepsis and the like), cutaneous nocardiosis (such as mycetoma, lymphocutaneous infections and the like), skin infections (such as impetigo, erysipelas, cellulitis, skin ulcers, secondary cutaneous involvement with disseminated disease and the like, scalded skin syndrome); leprosy, mycobacterial lymphadenitis, kidney infections, malacoplakia, puerperal sepsis, bloodstream infections (such as typhoid and the like), anthrax, plagues (such as bubonic plague, pneumonic plague, primary and secondary septicemic plague and the like); scarlet fever, rheumatic fever, cholera, Haverhill fever, Potomac fever, brucellosis, Carrion's disease, trench fever, bacillary epithelioid angiomatosis, leptospirosis, Lyme disease, rickettsiosis, Q fever, human monocytotropic ehrlichiosis, cat scratch disease, tularemia, pseudo-infections, legionellosis, noscoccomial infections (such as furuncles, postoperative wound infections of various sites and the like), erysipeloid, osteomyehtis, prostatitis, peritonitis, encephalitis, cerebrospinal infections, infection of cerebrospinal fluid shunt, meningoencephalitis, infection of the joints, prosthetic joint infections, septic arthritis, myonecrosis, echyma gangrenosum, cholecystitis, melioidosis, mastoiditis, epididymitis, bursitis, comamonas testosteroni infections, mastitis, cerebritis, abscesses (of muscle, urogenital tract, central nervous system, intra-abdominal, intracranial and the like), reproductive tract infections (such as vaginal infections, cervical lymphadenitis, gonorrhea, urethritis, endometritis, postpartum endometritis, perihepatitis, Chlamydia trachomatis infections, pelvic inflammatory disease, endocervical infections, salpingitis, pelvic peritonitis, tubo-ovarian abscesses, chancroid, amnionitis, chorioamnionitis, treponematosis and the like), toxic shock syndrome, meningococcemia, syphilis, postpartum fever, actinomycosis, sporatic bacterial enteritis, pancreatitis, haemophilus infections, epiglottitis, facial cellulitis, burns, diabetic foot, peritonitis, food poisoning, zoonosis, dermatophilosis, swine erysipelas, canine infections (such as pyoderma, reproductive tract infections and the like), avian borreliosis, egg peritonitis and the like.

In addition to being useful for human treatment, methods and compositions of the invention are useful for veterinary treatment of companion animals, exotic animals, farm animals and the like, particularly mammals.

In all embodiments of the invention an antibacterial agent and a selective COX-2 inhibitor are administered parenterally to a subject. An essential requirement for successful antibacterial therapy is that drug must reach a site of infection at concentrations near or higher than the minimal inhibitory concentrations and these concentrations must be maintained for a certain minimal time. There are significant differences among antibacterial agents in their ability to reach an infected site, and these are greater than the differences in their intrinsic antibacterial activities. Parenteral routes of administration offer numerous benefits over oral delivery in many situations, for a wide variety of drugs. One advantage of parenteral administration is that therapeutically effective blood serum concentrations of the drug are achieved in a shorter time than is achievable by other routes of administration. This results in more rapid onset of therapeutic action and more complete delivery to a site of infection, as compared with other routes of administration such as oral, transmucosal, transdermal, rectal and vaginal routes. This is especially true of intravenous injection, whereby the drug is placed directly in the bloodstream. Parenteral administration can also result in more predictable blood serum concentrations of a drug than oral administration, because losses in the gastrointestinal tract due to metabolism, partial or total degradation of the drug, binding to food, and other causes are eliminated. For similar reasons, parenteral administration often permits a significant dose reduction. The reduction in dosage for parenteral administration can often be as much as 50% of the dosage administered orally because of the improved bioavailability of the drug when administered parenterally. In addition, the effective use of some antibacterial agents requires continuous, controlled administration to achieve the desired effect. This type of prolonged delivery can be achieved by parenteral administration using mechanical perfusion devices that include a catheter and needle or by sustained release parenteral compositions. Parenteral administration is generally the preferred method of drug delivery in emergency situations, and is also useful in treating subjects with digestive tract illnesses or swallowing difficulties, as well as subjects who are uncooperative, unconscious, or otherwise unable or unwilling to accept oral medication.

Where a depot formulation is used, preferably the depot is other than a film coated or encapsulated liquid. Preferably a depot provides sustained plasma levels of the antibacterial agent and/or the selective COX-2 inhibitor for less than 50 days, for example for up to 30 days.

A parenterally deliverable pharmaceutical composition is also provided, comprising a vehicle that comprises at least one pharmaceutically acceptable excipient; said vehicle having dispersed therein an antibacterial agent in an antibacterially effective amount and a selective COX-2 inhibitor in an amount sufficient to provide systemic anti-inflammatory activity.

Such a composition is liquid or is reconstitutable with a parenterally acceptable diluent, e.g., water, to form a liquid. Liquid compositions of the invention include, but are not limited to, solutions, suspensions, slurries, emulsions, reconstituted compositions and the like. A pharmaceutically active agent can be present in the composition as drug particles, powders, granules, nanoparticles, microparticulates, microspheres, in lyophilized form, in dissolved form and the like. A suitable liquid composition of the invention can be aqueous based or oil based. Injectable preparations, for example sterile injectable aqueous or oleaginous suspensions, can be formulated according to known art using suitable dispersing or wetting agents and suspending agents. A sterile injectable preparation can be a sterile injectable solution or suspension in a nontoxic parenterally acceptable carrier, such as 1,3-butanediol.

Pharmaceutically acceptable aqueous carriers include, but are not limited to, Ringer's solution, isotonic sodium chloride solution, isotonic saline, sterile water and the like.

In addition, sterile fixed oils are conventionally employed as carriers. Pharmaceutically acceptable non-aqueous carriers include, but are not limited to, vegetable oils (such as cottonseed oil, corn oil, sesame oil, soybean oil, olive oil, fractionated coconut oils, peanut oil, sunflower oil, safflower oil, almond oil, avocado oil, palm oil, palm kernel oil, babassu oil, beechnut oil, linseed oil, rape oil and the like), mineral oils, synthetic oils and combinations thereof. Examples of fully saturated non-aqueous carriers include, but are not limited to, esters of medium to large chain fatty acids (such as fatty acid triglycerides with a chain length of about C₆ to about C₂₄). Mixtures of fatty acids are split from the natural oil (for example coconut oil palm kernel oil, babassu oil or the like) and are refined. In some embodiments, about C₈ to about C₁₂ fatty acid medium chain triglycerides are useful. An illustrative saturated non-aqueous carrier comprises capric acid (about 20% to about 45%) and caprylic acid (about 45% to about 80%). Other fully saturated non-aqueous carriers include, but are not limited to, saturated coconut oil (which typically includes a mixture of lauric, myristic, palmitic, capric and capric acids), including those sold under the Miglyol™ trademark from Huls and bearing trade designations 810, 812, 829 and 840). Also noted are the NeoBee™ products sold by Drew Chemicals. Isopropyl myristate is another example of a non-aqueous carrier useful in compositions of the invention. Examples of synthetic oils include triglycerides and propylene glycol diesters of saturated or unsaturated fatty acids having from 6 to 24 carbon atoms such as, for example hexanoic acid, octanoic (caprylic), nonanoic (pelargonic), decanoic (capric), undecanoic, lauric, tridecanoic, tetradecanoic (myristic), pentadecanoic, hexadecanoic (palmitic), heptadecanoic, octadecanoic (stearic), nonadecanoic, heptadecanoic, eicosanoic, heneicosanoic, docosanoic and lignoceric acids, and the like. Examples of unsaturated carboxylic acids include oleic, linoleic and linolenic acids, and the like. It is understood that the non-aqueous carrier can comprise the mono-, di- and triglyceryl esters of fatty acids or mixed glycerides and/or propylene glycol diesters wherein at least one molecule of glycerol has been esterified with fatty acids of varying carbon atom length. A non-limiting example of a “non-oil” of the present invention is polyethylene glycol.

Preferred non-aqueous carriers are selected from the group consisting of cottonseed oil, corn oil, peanut oil, sesame oil, soybean oil, olive oil, sunflower oil, safflower oil, almond oil, avocado oil, palm oil, palm kernel oil, babassu oil, beechnut oil, linseed oil, rape oil, mineral oil and fractionated coconut oil.

In one embodiment the carrier has not been modified to contain an increased level of oxidation products, through physical, chemical or mechanical means.

A suspension of the invention can be prepared by adding appropriate excipients as described herein, to a liquid vehicle and mixing to form a pharmaceutically acceptable carrier. Next an antibacterial agent and a selective COX-2 inhibitor are added to the carrier and mixed to form a uniform suspension.

Sterilization of a liquid composition of the invention can be achieved by any conventional method that preserves the biological activity of the composition, such as by filter sterilization. Compositions for intravenous administration are preferably terminally moist heat or steam heat sterilized. Terminal sterilization by gamma-irradiation can also be used for some parenteral compositions.

A reconstitutable composition applicable for use in the invention can be prepared substantially as described in International Patent Publication No. WO 02/80912, incorporated herein by reference.

Other parenterally deliverable compositions include, but are not limited to, conventional and in situ forming gels and implants and the like.

Methods for the preparation of in situ forming gels applicable for use with the invention are substantially described in the literature, for example in the patents individually cited below and incorporated herein by reference.

U.S. Pat. No. 4,861,760 to Mazuel & Friteyre.

U.S. Pat. No. 5,192,535 to Davis et al.

U.S. Pat. No. 5,587,175 to Viegas et al.

European Patent No. 0 424 043.

Optionally, administration of the therapeutic agents described herein can take place in further combination with other biologically active agents and non-drug therapies.

Antibacterial agents applicable for use according to the invention include any such agents that are effective for treatment and/or prevention of an infectious condition and/or complications associated therewith. Suitable antibacterial agents include, but are not limited to, beta-lactam antibacterials such as natural and synthetic penicillin type agents including penam penicillins (such as benzyl penicillin, phenoxymethyl penicillin, coxacillin, nafcillin, methicillin, oxacillin, amoxycillin, temocillin, ticarcillin and the like), penicillinase-stable penicillins, acylamino and carboxypenicillins (such as piperacillin, azlocillin, mezlocillin, carbenicillin, temocillin, ticarcillin and the like), and broader spectrum penicillins (such as streptomycin, neomycin, framycetin, gentamicin, apramycin, amikacin, spectinomycin, amoxycillin, ampicillin and the like), cephalosporins, macrolides (such as tylosin, tilmicosin, aivlosin, erythromycin, azithromycin, spiramycin, josamycin, kitasamycin and the like), lincosamides (such as lincomycin, clindamycin, pirlimycin and the like), pleuromutilins (such as tiamulin, valnemulin and the like), polypeptides, glycopeptides (such as vancomycin and the like), polymixins (such as polymixin B, polymixin E and the like), sulfonamides (such as sulfamethazine, sulfadiazine, sulfatroxazole, sulfamethoxypyridazine, sulfanilamide, sulfamethoxazole, sulfisoxazole, sulfamethizole, silver sulfadiazine, mafenide and the like, alone or in combination with trimethoprim), chloramphenicol, thiamphenicol, florfenicol, tetracycline type agents (such as tetracycline, chlortetracycline, oxytetracycline, domeclocycline, doxycycline, minocycline and the like), quinolones and fluoroquinolones (such as ciprofloxacin, enoxacin, grepafloxacin, levofloxacin, lomefloxacin, norfloxacin, ofloxacin, sparfloxacin, trovafloxacin, cinocacin, nalidixic acid and the like), tiamulin, colistin, meropenem, sulbactam, tazobactam, methacycline, pyrimethamine, sulfacetamide, oxazolidinones, e.g., eperezolid, linezolid, N-((5S)-3-(3-fluoro-4-(4-(2-fluoroethyl)-3-oxy-1-piperazinyl)phenyl-2-oxy-5-oxazolidinyl)methyl)acetamide, (S)-N-((3-(5-(3-pyridyl)thiophen-2-yl)-2-oxy-5-oxazolidinyl)methyl)acetamide, (S)-N-((3-(5-(4-pyridyl)pyrid-2-yl)-2-oxy-5-oxazolidinyl)methyl)acetamide hydrochloride, 2,2-difluoro-N-({(5S)-3-[3-fluoro-4-(4-glycoloylpiperazin-1-yl)phenyl]-2-oxo-1,3-oxazolidin-5-yl}methyl)ethanethioamide and the like, aminoglycosides (kanamycin, tobramycin, netilmicin and the like), aminocyclitols, amphenicol, ansamycin, carbaphenem, cephamycin, rifampicin, monobactam, oxacephem, streptogramins (such as quinupristin, dalfopristin and the like), cycloserines, mupirocin, urea hydroxamates, folic acid analogs (such as trimethoprim and the like), antibiotic-type antineoplastic agents (such as aclarubicin, actinomycin D, actinoplanone, aeroplysinin derivative, Nippon Soda anisomycins, anthracycline, azino-micyin-A, busucaberin, bleomycin sulfate, bryostatin-1, calichemycin, chromoximycin, dactinomycin, daunorubicin, ditrisarubicin B, doxorubicin, doxorubicin-fibrinogen, elsamicin-A, epirubicin, erbstatin, esorubicin, esperamicin-A1b, fostriecin, glidobactin, gregatin-A, grincamycin, herbimycin, idarubicin, illudins, kazusamycin, kesarirhodins, menogaril, mitomycin, mitoxantorone, mutamycin, mycophenolate mofetil, neoenactin, oxalysine, oxaunomycin, peplomycin, pilatin, pirarubicin, porothramycin, pyrindamycin A, rapamycin, rhizoxin, rodorubicin, sibanomicin, siwenmycin, sorangicin-A, sparsomycin, steffimycin B, talisomycin, terpentecin, thrazine, tricrozarin A, zorubicin and the like), systemic antibacterials (such as 2,4-diaminopyrimidine), nitrofuran sulfones, marbofloxacin and the like, and combinations thereof.

It should be understood that any reference herein to a particular drug compound includes tautomers, stereoisomers, enantiomers, salts, hydrates and prodrugs of that compound and is not specific to any one solid state form of the drug.

Preferred antibacterial agents applicable for use according to the invention are cephalosporins including, but not limited to, ceftiofur hydrochloride, ceftiofur free acid, e.g., ceftiofur crystalline free acid, ceftiofur sodium, other ceftiofur salts, cephalexin, cephradine, cefquinome, cephacetrile, cephalonium, cefuroxime, cefazidime, cefoperazone, sodium cephemethcarboxylate, cephem heptahydrate, cephalosporin di- or tri-hydrate, cephadroxil monohydrate, cephazolin sodium monohydrate, cefiximine, ceftaxime, ceftizoxime, ceftriaxone, o-formylcefamandole, salts of 3-acetoxymethyl-7-(iminocetamido)-cephalosporanic acid derivatives, monohydrate of 7-(D-alpha-amino-alpha-(p-hydroxyphenyl)acetamino)-3-methyl-3-cephem-1-carboxylic acid, hydrochloride salt of syn-7-((2-amino-1-thiazolyl)(methoxyimino)acetyl)amino)-3-methyl-3-cephem-4-carboxylic acid, cephem acid addition salts, (pivaloyloxy)methyl 7-beta-(2-(2-amino-4-thiazolyl)acetamido)-3-(((1-(2-(dimethylamino)ethyl)-1H-tetraazol-5-yl)thio)methyl)-3-cephem-4-carboxylate, cephalexin, cephalexin monohydrate, 7-(D-2-naphthyglycylamino)-3-methyl-3-cephem-4-carboxylic acid tetrahydrate and the like. The most preferred cephalosporins for use according to the present invention are ceftiofur and pharmaceutically acceptable salts thereof. Especially preferred are ceftiofur free acid, most especially in crystalline form, and ceftiofur hydrochloride.

Where the antibacterial substance is ceftiofur or a salt thereof, a preferred concentration range in a composition of the invention is about 1 to about 1000 mg/ml, more preferably about 5 to about 750 mg/ml, and still more preferably about 10 to about 100 mg/ml. For antibacterial substances other than ceftiofur, suitable concentration ranges that are antibacterially equivalent can be determined by one of skill in the art based upon published data.

Examples of selective COX-2 inhibitors applicable for use according to the invention include, but are not limited to, the compounds described below and include tautomers, stereoisomers, enantiomers, salts, hydrates, prodrugs and combinations thereof. Any such selective COX-2 inhibitory drug or prodrug known in the art can be used.

A preferred selective COX-2 inhibitory drug useful herein is a compound of formula (I):

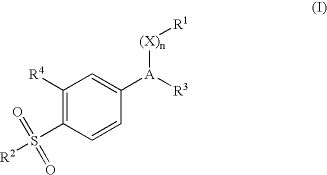

or a prodrug or pharmaceutically acceptable salt thereof, wherein:

A is a substituent selected from partially unsaturated or unsaturated heterocyclyl and partially unsaturated or unsaturated carbocyclic rings, preferably a heterocyclyl group selected from pyrazolyl, furanonyl, isoxazolyl, pyridinyl, cyclopentenonyl and pyridazinonyl groups;

X is O, S or CH₂;

n is 0 or 1;

R¹ is at least one substituent selected from heterocyclyl, cycloalkyl, cycloalkenyl and aryl, and is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

R² is methyl, amino or aminocarbonylalkyl;

R³ is one or more radicals selected from hydrido, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, arylsulfonyl and N-alkyl-N-arylaminosulfonyl, R³ being optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio; and

R⁴ is selected from hydrido and halo.

A particularly preferred group of selective COX-2 inhibitory drugs are compounds having the formula (II):

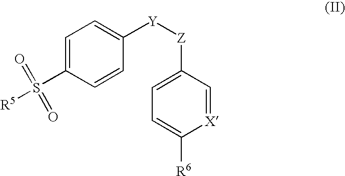

where R⁵ is a methyl or amino group, R⁶ is hydrogen or a C₁₋₄ alkyl or alkoxy group, X′ is N or CR⁷ where R⁷ is hydrogen or halogen, and Y and Z are independently carbon or nitrogen atoms defining adjacent atoms of a five- to six-membered ring that is optionally substituted at one or more positions with oxo, halo, methyl or halomethyl groups, or an isomer, tautomer, pharmaceutically-acceptable salt or prodrug thereof. Preferred such five- to six-membered rings are cyclopentenone, furanone, methylpyrazole, isoxazole and pyridine rings substituted at no more than one position.

Another particularly preferred group of selective COX-2 inhibitory drugs are compounds having the formula (III):

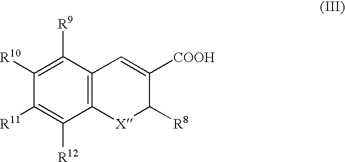

where X″ is O, S or N-lower alkyl; R⁸ is lower haloalkyl; R⁹ is hydrogen or halogen; R¹⁰ is hydrogen, halogen, lower alkyl, lower alkoxy or haloalkoxy, lower aralkylcarbonyl, lower dialkylaminosulfonyl, lower alkylaminosulfonyl, lower aralkylaminosulfonyl, lower heteroaralkylaminosulfonyl, or 5- or 6-membered nitrogen-containing heterocyclosulfonyl; and R¹¹ and R¹² are independently hydrogen, halogen, lower alkyl, lower alkoxy or aryl; and pharmaceutically acceptable salts thereof.

A particularly useful compound of formula (III) is (S)-6,8-dichloro-2-(trifluoromethyl)-2H-1-benzopyran-3-carboxylic acid.

Another particularly preferred group of selective COX-2 inhibitory drugs are 5-alkyl-2-arylaminophenylacetic acids and derivatives thereof. Particularly useful compounds of this class are lumiracoxib and pharmaceutically acceptable salts thereof.

Illustratively, celecoxib, deracoxib, valdecoxib, parecoxib, rofecoxib, etoricoxib, lumiracoxib, 2-(3,5-difluorophenyl)-3-[4-(methylsulfonyl)phenyl]-2-cyclopenten-1-one, (S)-6,8-dichloro-2-(trifluoromethyl)-2H-1-benzopyran-3-carboxylic acid, 2-(3,4-difluorophenyl)-4-(3-hydroxy-3-methyl-1-butoxy)-5-[4-(methylsulfonyl)phenyl]-3-(2H)-pyridazinone, 4-[5-(4-fluorophenyl)-3(trifluoromethyl)-1H-pyrazol-1-yl]benzenesulfonamide, 4-[5-(phenyl)-3-(trifluoromethyl)-1H-pyrazol-1-yl]benzenesulfonamide, and their salts, more particularly celecoxib, deracoxib, valdecoxib, parecoxib and its salts, rofecoxib, etoricoxib, lumiracoxib, 4-[5-(4-fluorophenyl)-3-(trifluoromethyl)-1H-pyrazol-1-yl]benzenesulfonamide, and 4-[5-(phenyl)-3-(trifluoromethyl)-1H-pyrazol-1-yl]benzenesulfonamide are useful in the method and composition of the invention.

Valdecoxib used in compositions of the invention can be prepared by any known process, for example in the manner set forth in U.S. Pat. No. 5,633,272 to Talley et al. Parecoxib and salts thereof used in compositions of the invention can be prepared by any known process, for example in the manner set forth in U.S. Pat. No. 5,932,598 to Talley et al. Rofecoxib used in compositions of the invention can be prepared by any known process, for example in the manner set forth in U.S. Pat. No. 5,474,995 to Ducharme et al. Etoricoxib used in compositions of the invention can be prepared by any known process, for example in the manner set forth in International Patent Publication No. WO 98/03484. 2-(3,5-Difluorophenyl)-3-[4-(methylsulfonyl) phenyl]-2-cyclopenten-1-one used in compositions of the invention can be prepared by any known process, for example in the manner set forth in European Patent No. 0 863 134. Deracoxib used in compositions of the invention can be prepared by any known process, for example in the manner set forth in U.S. Pat. No. 5,760,068 to Talley et al. 2-(3,4-Difluorophenyl)-4-(3-hydroxy-3-methyl-1-butoxy)-5-[4-(methylsulfonyl)phenyl]-3-(2H)-pyridazinone used in compositions of the invention can be prepared by any known process, for example in the manner set forth in International Patent Publication No. WO 00/24719. Other selective COX-2 inhibitory drugs can be prepared by any known process, including processes set forth in patent publications disclosing such drugs; for example in the case of celecoxib in above-cited U.S. Pat. No. 5,466,823 or in U.S. Pat. No. 5,892,053 to Zhi et al. All patents and publications cited above are incorporated herein by reference.

A preferred concentration range for a selective COX-2 inhibitor in a composition of the invention is about 0.01 to about 1000 mg/ml, more preferably about 0.1 to about 750 mg/ml, and still more preferably about 5 to about 250 mg/ml.

A composition of the invention can be admixed with any conventional pharmaceutical additive that does not deleteriously react with other ingredients of the composition. Such additives include, but are not limited to, diluents, antioxidants, preservatives, stabilizers, thickening agents, suspending agents, dispersing agents, solubilization agents, isotonic agents, buffering agents, wetting agents, lubricants, emulsifiers, salts for influencing osmotic pressure, coloring agents, alcohols, other surfactants and conventional pharmaceutical additives and the like, and combinations thereof.

Illustrative excipients include without limitation tocopherols, ascorbyl palmitate, butyl hydroxyanisole, butyl hydroxytoluene, benzoic acid and derivatives thereof, ascorbic acid and salts thereof, e.g., sodium ascorbate, methionine, ethylenediamine, sodium bisulfite, sulfur dioxide, maleic acid, propyl gallate, parabens, chlorbutanol, phenol, sorbic acid and salts thereof, thimerosal, colloidal silica, petrolatum, aluminum stearate, magnesium stearate, talc, sorbitol, dextran, dextrose, lanolin, ceresin, spermaceti, chitosan, paraffin, cellulose ether polymers, starch, propylene glycol, dipropylene glycol, hexylene glycol, polyethylene glycol, ethanol, carrageenan, 12-hydroxystearin, polyvinylpyrrolidone, hydroxyethylpropylcellulose, hydroxyethylcellulose, hydroxypropylmethylcellulose, natural gums such as guar, xanthan and tragacanth gums, silicic acid, carbohydrates, cellosolves such as methyl cellosolve and ethyl cellosolve, vegetable oils and waxes containing at least about 12 carbons in a straight chain, e.g., olive oil and castor oil, trisodium orthophosphate, sodium bicarbonate, N-methylglucamine, L(+)-lysine, L(+)-arginine, acetic acid, boric acid, citric acid, lactic acid, phosphoric acid, hydrochloric acid, sodium hydroxide, sodium phosphate, potassium phosphate, potassium citrate, sodium lactate, mono-, di- and triethanolamines, 2-amino-2-(hydroxymethyl)-1,3-propanediol, tris-hydroxymethylaminomethane, citrate/dextrose, sodium bicarbonate, ammonium chloride, esters such as amyl acetate, ethyl acetate and benzyl benzoate and the like, and combinations thereof.

It will be appreciated that the preferred amounts of compositions to be administered in a specific case will vary according to the specific composition being utilized, the mode of application, the particular situs and organism being treated, and other factors. Dosages for a given purpose can be determined using conventional considerations, for example, by customary comparison of the differential activities of the subject compositions and of a known agent, e.g., by means of an appropriate conventional pharmaceutical protocol.

An illustrative suspension of the invention containing an antibacterial agent, e.g., ceftiofur hydrochloride, and a selective COX-2 inhibitor, e.g., deracoxib, has the following composition:

| antibacterial agent | 1-150 mg/ml |
|---|---|
| selective COX-2 inhibitor | 1-350 mg/ml |
| vehicle | 0.5-99% |

(all percentages are weight/volume).

### EXAMPLES

The following examples illustrate aspects of the present invention but should not be construed as limitations.

### Example 1

An antibacterial suspension to be administered by subcutaneous injection is prepared having the following composition:

| | |
|---|---|
| ceftiofur hydrochloride (micronized) | 100 mg/ml |
| Labrafil ™ M-1944CS | 200 mg/ml |
| cottonseed oil NF | q.s. |

The Labrafil™ M-1944CS and cottonseed oil are mixed in a manufacturing tank to form the vehicle. Ceftiofur hydrochloride is added to the resulting vehicle and mixed to form a uniform suspension. The suspension is screened and filled into 25 ml glass vials. The packaged product is terminally sterilized by gamma irradiation at a dose of 25-40 kGy.

A parecoxib sodium solution in 0.9% sodium chloride for injection USP to be administered by intravenous injection is prepared by reconstituting a lyophilized formulation as described in International Patent Publication No. WO 02/80912.

The ceftiofur hydrochloride suspension and the parecoxib sodium solution are administered to a subject subcutaneously and intravenously respectively, at a dose of 4 mg ceftiofur hydrochloride/kg body weight/day and 0.6 mg parecoxib sodium/kg of body weight/day. The compositions are effective in treatment of otitis externa.

### Example 2

A suspension to be administered by intramuscular injection is prepared having the following composition:

| | |
|---|---|
| ceftiofur crystalline free acid (micronized) | 50 mg/ml |
| deracoxib | 62.5 mg/ml |
| corn oil NF | q.s. |

The ceftiofur crystalline free acid and the deracoxib are added to the corn oil in a manufacturing tank and mixed to form a uniform suspension. The suspension is screened and filled into 20 ml glass vials. The packaged product is terminally sterilized by gamma irradiation at a dose of 25-40 kGy.

The above suspension is administered to a subject by intramuscular injection at a dose of 4 mg ceftiofur/kg body weight/day and 5 mg deracoxib/kg body weight/day. The suspension is effective in treatment of skin infections.

### Example 3

A suspension to be administered by subcutaneous injection is prepared having the following composition:

| | |
|---|---|
| ceftiofur hydrochloride (micronized) | 75 mg/ml |
| celecoxib | 75 mg/ml |
| Labrafil ™ WL-2609BS | 50 mg/ml |
| cottonseed oil NF | q.s. |

The cottonseed oil and Labrafil™ WL-2609BS are added to a manufacturing tank and mixed to form the vehicle. The ceftiofur hydrochloride and celecoxib are added to the resulting vehicle and mixed to form a uniform suspension. The suspension is screened and filled into 20 ml high glass vials. The packaged product is terminally sterilized by gamma irradiation at a dose of 25-40 kGy.

The above suspension is administered to by subcutaneous injection to a subject at a dose of 4 mg ceftiofur hydrochloride/kg body weight/day and 4 mg celecoxib/kg body weight/day. The suspension is effective in treatment of wound infections.

The invention having been described in detail and by reference to the preferred embodiments thereof, it will be apparent that modifications and variations are possible without departing from the scope of the appended claims.

What is claimed is:

1. A method of treatment and/or prevention of an non-ocular infective condition having an inflammatory component, the method comprising parenterally administering to a subject an antibacterial agent in an antibacterially effective amount, in combination therapy with a selective COX-2 inhibitor in an amount sufficient to provide systemic anti-inflammatory activity.

2. The method of claim 1 wherein the infective condition is mediated by a gram positive organism.

3. The method of claim 1 wherein the infective condition is mediated by a gram negative organism.

4. The method of claim 1 wherein the infective condition is selected from the group consisting of bacterial soft tissue infections, infections of the respiratory system including upper respiratory tract infections, sinusitis, ear infections, otolaryngological infections, infections of the gastrointestinal tract; bacterial meningitis; infection related to abdominal trauma; pyelonephritis; nocardial pulmonary infections; infections of the cardiovascular system including endocarditis, myocarditis and intravascular catheter related infections; synovitis; infections arising from wounds including external wounds, injuries, scalds, bites, sternal wounds, mammaplasty wounds, surgical procedures, etc.; bacteremia, septicemia, acute glomerulonephritis, neonatal infections, diphtheria, intracellular and extracellular bacterial infections; urinary tract infections, cutaneous nocardiosis, skin infections; leprosy, mycobacterial lymphadenitis, kidney infections, malacoplakia, puerperal sepsis, bloodstream infections, anthrax, plagues; scarlet fever, rheumatic fever, cholera, Haverhill fever, Potomac fever, brucellosis, Carrion's disease, trench fever, bacillary epithelioid angiomatosis, leptospirosis, Lyme disease, rickettsiosis, Q fever, human monocytotropic ehrlichiosis, cat scratch disease, tularemia, pseudo-infections, legionellosis, noscoccomial infections, erysipeloid, osteomyelitis, prostatitis, peritonitis, encephalitis, cerebrospinal infections, infection of cerebrospinal fluid shunt, meningoencephalitis, infection of the joints, prosthetic joint infections, septic arthritis, myonecrosis, echyma gangrenosum, cholecystitis, melioidosis, mastoiditis, epididymitis, bursitis, comamonas testosteroni infections, mastitis, cerebritis, abscesses, reproductive tract infections, toxic shock syndrome, meningococcemia, syphilis, postpartum fever, actinomycosis, sporatic bacterial enteritis, pancreatitis, Haemophilus infections, epiglottitis, facial celulitis, burns, diabetic foot, peritonitis, food poisoning, zoonosis, dermatophilosis, swine erysipelas, canine infections, avian borreliosis and egg peritonitis.

5. The method of claim 1 wherein the antibacterial agent and the selective COX-2 inhibitor are each administered by a parenteral route independently selected from the group consisting of intravenous, intramuscular and subcutaneous routes.

6. The method of claim 1 wherein the antibacterial agent and the selective COX-2 inhibitor are each administered intravenously.

7. The method of claim 1 wherein the antibacterial agent and the selective COX-2 inhibitor are administered as a single pharmaceutical composition comprising said antibacterial, said selective COX-2 inhibitor and a pharmaceutically acceptable vehicle.

8. The method of claim 1 wherein the antibacterial agent is selected from the group consisting of natural and synthetic penicillin-type antibiotics, cephalosporins, macrolides, lincosamides, pleuromutilins, polypeptides, polymixins, sulphonamides, chloramphenicol, thiamphenicol, florfenicol, tetracycline-type antibiotics, quinolones, fluroquinolones, tiamulin, colistin, domeclocycline, mafenide, methacycline, ofloxacin, pyrimethamine, silver sulfadiazine, sulfacetamide, sulfisoxazole, tobramycin, vanemulin, oxazolidinones, glycopeptides, amino glycosides and aminocyclitols, amphenicol, ansamycin, carbaphenem, cephamycin, vancomycin, monobactam, oxacephem, systemic antibacterials, nitrofuran sulfones, marbofloxacin, and tautomers, stereoisomers, enantiomers, salts, hydrates and prodrugs thereof.

9. The method of claim 1 wherein the antibacterial agent is an oxazolidinone selected from the group consisting of eperezolid, linezolid, N-((5S)-3-(3-fluoro-4-(4-(2-fluoroethyl)-3-oxy-1-piperazinyl)phenyl-2-oxy-5-oxazolidinyl)methyl)acetamide, (S)-N-((3-(5-(3-pyridyl)thiophen-2-yl)-2-oxy-5-oxazolidinyl)methyl)acetamide, 2,2-difluoro-N-({(5S)-3-[3-fluoro-4-(4-glycoloylpiperazin-1-yl)phenyl]-2-oxo-1,3-oxazolidin-5-yl}methyl)ethanethioamide and (S)-N-((3-(5-(4-pyridyl)pyrid-2-yl)-2-oxy-5-oxazolidinyl)methyl)acetamide hydrochloride.

10. The method of claim 1 wherein the antibacterial agent is a cephalosporin.

11. The method of claim 1 wherein the antibacterial agent is a cephalosporin selected from the group consisting of ceftiofur, cephalexin, cephradine, cefquinome, cephacetrile, cephalonium, cefuroxime, cefazidime, cefoperazone, sodium cephemethcarboxylate, cephem, cephadroxil, cephazolin sodium, cefiximine, ceftaxime, ceftizoxime, ceftriaxone, o-formylcefamandole, salts of 3-acetoxymethyl-7-(iminocetamido)-cephalosporanic acid derivatives, 7-(D-α-amino-α-(p-hydroxyphenyl)acetamino)-3-methyl-3-cephem-1-carboxylic acid, hydrochloride salt of syn-7-((2-amino-1-thiazolyl)(methoxyimino)acetyl)amino)-3-methyl-3-cephem-4-carboxylic acid, cephem acid, (pivaloyloxy)methyl-7-beta-(2-(2-amino-4-thiazolyl)acetamido)-3-(((1-(2-(dimethylamino)ethyl)-1H-tetraazol-5-yl)thio)methyl)-3-cephem-4-carboxylate, cephalexin, 7-(D-2-naphthyglycylamino)-3-methyl-3-cephem-4-carboxylic acid, and tautomers, stereoisomers, enantiomers, salts, hydrates and prodrugs thereof.

12. The method of claim 1 wherein the antibacterial agent is ceftiofur or a pharmaceutically acceptable salt thereof.

13. The method of claim 1 wherein the antibacterial agent is ceftiofur hydrochloride.

14. The method of claim 1 wherein the antibacterial agent is ceftiofur crystalline free acid.

15. The method of claim 12 wherein the antibacterial agent is administered in a pharmaceutical composition adapted for parenteral administration.

16. The method of claim 15 wherein the antibacterial agent is present in the composition at a concentration of about 1 to about 1000 mg/ml.

17. The method of claim 15 wherein the antibacterial agent is present in the composition at a concentration of about 5 to about 750 mg/ml.

18. The method of claim 15 wherein the antibacterial agent is present in the composition at a concentration of about 10 to about 100 mg/ml.

19. The method of claim 1 wherein the selective COX-2 inhibitor is a compound having the formula 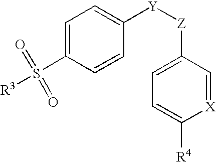
where R³ is a methyl, amino or imide group, R⁴ is hydrogen or a C₁₄ alkyl or alkoxy group, X is N or CR⁵ where R⁵ is hydrogen or halogen, and Y and Z are independently carbon or nitrogen atoms defining adjacent atoms of a five- to six-membered ring that is unsubstituted or substituted at one or more positions with oxo, halo, methyl or halomethyl groups.

20. The method of claim 1 wherein the selective COX-2 inhibitor is selected from the group consisting of deracoxib, parecoxib, celecoxib, valdecoxib, rofecoxib, etoricoxib, lumiracoxib, 2-(3,5-difluorophenyl)-3-[4-(methylsulfonyl)phenyl]-2-cyclopenten-1-one, (S)-6,8-dichloro-2-(trifluoromethyl)-2H-1-benzopyran-3-carboxylic acid, 2-(3,4-difluorophenyl)-4-(3-hydroxy-3-methyl-1-butoxy)-5-[4-(methylsulfonyl)phenyl]-3-(2H)-pyridazinone, 4-[5-(4-Fluorophenyl)-3-(trifluoromethyl)-1H-pyrazol-1-yl]benzenesulfonamide, 4-[5-(phenyl)-3-(trifluoromethyl)-1H-pyrazol-1-yl]benzenesulfonamide, and salts and prodrugs thereof.

21. The method of claim 1 wherein the selective COX-2 inhibitor is deracoxib.

22. The method of claim 1 wherein the selective COX-2 inhibitor is parecoxib or a salt thereof.

23. The method of claim 1 wherein the selective COX-2 inhibitor is celecoxib.

24. The method of claim 1 wherein the selective COX-2 inhibitor is valdecoxib.

25. The method of claim 1 wherein the selective COX-2 inhibitor is administered in a composition adapted for parenteral administration.

26. The method of claim 25 wherein the selective COX-2 inhibitor is present in the composition at a concentration of about 0.01 to about 1000 mg/ml.

27. The method of claim 25 wherein the selective COX-2 inhibitor is present in the composition at a concentration of about 0.1 to about 750 mg/ml.

28. The method of claim 25 wherein the selective COX-2 inhibitor is present in the composition at a concentration of about 5 to 250 mg/ml.

29. The method of claim 1 wherein said administration effects rapid delivery of the antibacterial agent and the selective COX-2 inhibitor to a site of said infection.

30. A parenterally deliverable pharmaceutical composition comprising a vehicle that comprises at least one pharmaceutically acceptable excipient; said vehicle having dispersed therein an antibacterial agent in an antibacterially effective amount and a selective COX-2 inhibitor in an amount sufficient to provide systemic anti-inflammatory activity, wherein said composition upon parenteral administration to a subject is effective in treatment and/or prevention of an infective condition having an inflammatory component.

31. The composition of claim 30 that further comprises at least one excipient selected from the group consisting of diluents, antioxidants, preservatives, stabilizers, thickening agents, suspending agents, dispersing agents, solubilization agents, isotonic agents, buffering agents, wetting agents, lubricants, emulsifiers, salts for influencing osmotic pressure, coloring agents, alcohols, other surfactants and conventional pharmaceutical additives.
